(19) United States
(12) Patent Application Publication
(10) Pub. No.: US 2025/0295402 A1
(43) Pub. Date: Sep. 25, 2025

(54) MEDICAL SYSTEMS, DEVICES, AND METHODS FOR PROVIDING TRACTION TO TISSUE

(71) Applicant: Boston Scientific Scimed, Inc., Maple Grove, MN (US)

(72) Inventors: Rosangel Aimee RAMOS ESPINOZA, Boston, MA (US); Kensuke HAYASHI, Penang (MY); Scott Edward CORBEIL, Litchfield, NH (US)

(73) Assignee: Boston Scientific Scimed, Inc., Maple Grove, MN (US)

(21) Appl. No.: 19/086,478

(22) Filed: Mar. 21, 2025

Related U.S. Application Data
(60) Provisional application No. 63/568,560, filed on Mar. 22, 2024.

(51) Int. Cl. A61B 17/02; A61B 17/00; A61B 17/29

(52) U.S. Cl. CPC A61B 17/0218; A61B 17/29; A61B 2017/00269; A61B 2017/00296; A61B 2017/2926; A61B 2017/2944

(57) ABSTRACT

A medical system may comprise a shaft, a first grasping device including a first pair of jaws, and a second grasping device coupled to the shaft, including a second pair of jaws.

### CROSS-REFERENCE TO RELATED APPLICATIONS

This application claims the benefit of priority of U.S. Provisional Patent Application No. 63/568,560, filed on Mar. 22, 2024, the entirety of which is incorporated herein by reference.

### TECHNICAL FIELD

Various aspects of this disclosure relate generally to medical systems, devices, and methods for providing traction to tissue. In particular, aspects of the disclosure relate to systems, devices, and methods for applying traction to a target tissue to facilitate resection of the target tissue.

### BACKGROUND

Endoscopic procedures may involve removing a lesion from a body lumen. For example, endoscopic submucosal dissection (ESD) utilizes endoscopic tools to remove cancerous or other lesions from the gastrointestinal tract. An instrument, such as an electrosurgical knife, may be passed through a working channel of an endoscope in order to cut and remove the lesion. In some procedures, a lifting agent may be injected under the lesion prior to removing the lesion, so as to facilitate access by the electrosurgical knife or other instrument. A need exists for applying traction or tension to a lesion or other portion of a body lumen to facilitate medical procedures, such as ESD.

### SUMMARY

Each of the aspects disclosed herein may include one or more of the features described in connection with any of the other disclosed aspects. Aspects of the disclosure may relate to systems, devices, and methods for applying traction to a tissue.

In an example, a medical device may comprise a shaft and a first grasping device coupled to the shaft that may include a first pair of jaws. The medical device may also comprise a second grasping device coupled to the shaft and include a second pair of jaws.

According to some aspects, the medical device may include the first grasping device on a distalmost end of the shaft and the second grasping device may extend radially outward from the shaft. A handle may be configured to actuate the first grasping device and the second grasping device. The handle may include a first actuator that is configured to actuate the first grasping device and the second grasping device. A first wire may extend through the shaft and couple to the first grasping device and a second wire may extend through the shaft and couple to the second grasping device. The shaft may be a first shaft, and the second grasping device is further coupled to a distalmost end of a second shaft and the second shaft may extend through the first shaft. The first grasping device may be configured to grasp a target tissue, and the second grasping device may be configured to grasp a wall of a body lumen. In a configuration in which the first grasping device grasps a target tissue and the second grasping device grasps a wall of a body lumen, traction is applied to the target tissue.

Further, the medical device may be a first medical device and the shaft may be a first shaft, and the system may further include a second medical device having a handle and a second shaft. The first shaft of the first medical device may be configured to be external to the second shaft of the second medical device. The second medical device may be a scope device. The second shaft may include a guide for coupling to the first shaft. The guide may include a ring, a prong, or a hook. The guide may be coupled to a coupler and the coupler may extend radially around the first shaft.

The disclosure also includes a medical device comprising a handle, a first shaft, and a guide extending radially outward from the first shaft. The guide may include a ring, a hook, or a prong, and the guide may be configured to receive a second shaft of an accessory instrument.

The disclosure also includes a medical device that may include a first anchor, the first anchor may include a stem and a bar. The bar may extend approximately perpendicularly to the stem, such that the anchor has a “T” shape. The medical device may also include a second anchor and a tether that may couple the first anchor and the second anchor. According to some aspects, the second anchor may have a substantially rectangular shape.

### BRIEF DESCRIPTION OF THE DRAWINGS

The accompanying drawings, which are incorporated in and constitute a part of this specification, illustrate aspects this disclosure, and together with the description, serve to explain the principles of the disclosure.

FIG. 1 depicts an exemplary system for applying traction to a tissue.

FIGS. 2A-2C depicts a distal portion of another exemplary system.

FIG. 3 depicts another system for applying traction to tissue.

FIG. 4 depicts an exemplary device for applying traction to a tissue.

### DETAILED DESCRIPTION

It may be understood that both the foregoing general description and the following detailed description are exemplary and explanatory only and are not restrictive of the invention, as claimed. As used herein, the terms “comprises,” “comprising,” “has,” “having,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from a user, and the term “proximal” refers to a direction toward a user. The term “exemplary” is used in the sense of “example,” rather than “ideal.” The term “approximately,” or like terms (e.g., “substantially”), includes values+/−10% of a stated value. Unless otherwise stated, ranges disclosed herein include the end points of the ranges. The use of ordinals (e.g., first, second, third, fourth) herein is for convenient reference to different examples and does not imply any ordered or other type of relationship among identified elements. The ordinal applied to a given element is arbitrary, and any alternative ordinal may be used with the element.

A system for applying traction to tissue may include an endoscope and a medical instrument. The medical instrument may be external to the endoscope. In one example, the medical instrument may include a first grasping device and a second grasping device that may apply traction to target tissue. The handle of the first medical instrument may include a first actuator and a second actuator configured to actuate the first grasping device and the second grasping device. The first and second grasping devices may be configured to grasp and/or lift the tissue.

In another example, an endoscope may include a guide device on an outer surface of the endoscope in order to receive and guide a medical instrument and to couple (e.g., removably couple) the medical instrument to the endoscope. The medical instrument may be used to apply traction to a tissue but is not so limited.

The disclosure also relates to devices for applying traction to a target tissue. For example, a device may include a plurality of anchors (e.g., a tag and a bar) for coupling to a target tissue and an opposing body lumen wall for applying traction to the target tissue. In another example, a device may include a plurality of clips tethered with one or more bands. The clips may be delivered to a target tissue and/or to opposing walls of body lumens to apply traction to the target tissue.

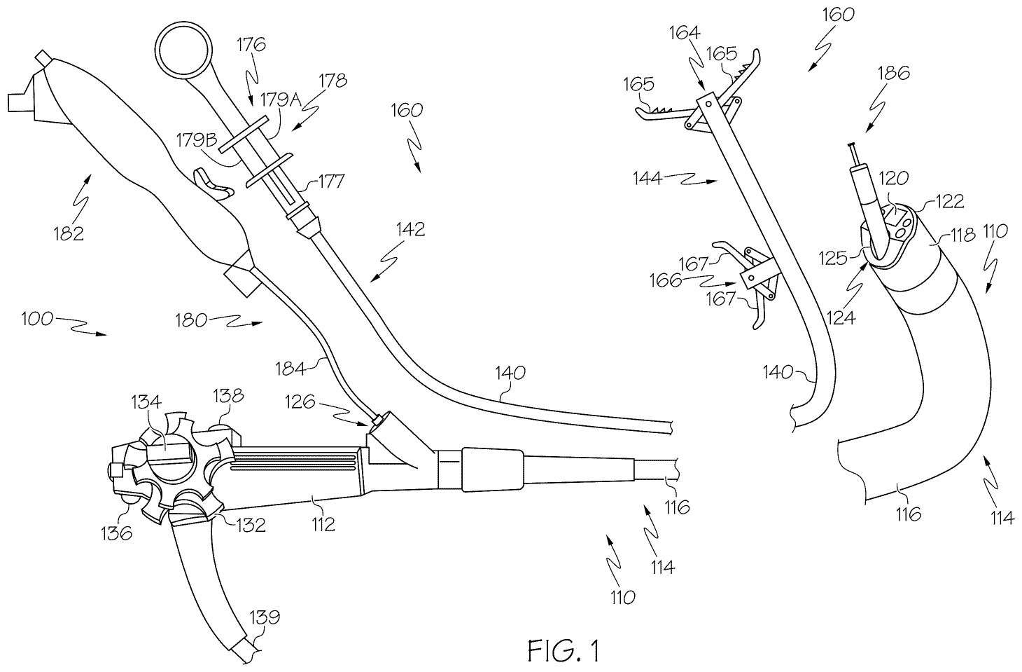

FIG. 1 depicts an exemplary medical system 100. The medical system 100 may include an endoscope 110 (a medical device) and a first medical instrument 160 that may be external to the endoscope 110. As described herein, the first medical instrument 160 may include a first grasping device 164 and a second grasping device 166 that may apply traction and/or tension to a target tissue (e.g., to a lesion or to an area neighboring a lesion) and/or may create a tissue flap.

The endoscope 110 may have any of the features of any endoscope known in the art. For example, the endoscope 110 may include a handle 112 and an insertion portion 114. The insertion portion 114 may have a shaft 116 and a distal tip 118. The distal tip 118 may include one or more imaging devices 120 and/or lighting elements 122. The imaging device 120 may include a camera and/or other optical device(s) for transmitting an image from a body lumen to a user (e.g., optical fiber, lens, image sensor, etc.). The lighting elements 122 may include any element that may deliver light to a procedure site, such as light emitting diode(s), optical fibers, or other types of light sources. The distal tip 118 may also include a distal opening 125 of a working channel 124. The working channel 124 may extend from the handle, through the shaft, to distal opening 125. The handle 112 may include a port 126 that defines a proximal opening of the working channel 124.

The handle 112 may include one or more control elements for controlling aspects of the insertion portion 114. For example, the handle 112 may include one or more of the steering actuators 132 (e.g., knobs or levers) and/or one or more of the braking actuators 134 (e.g., knobs or levers). The steering actuators 132 may steer a distal portion of the shaft 116 (e.g., by manipulating an articulation joint of the shaft 116). The braking actuators 134 may help to inhibit steering of the distal portion of the shaft 116. The handle 112 may also include one or more actuators 136 for controlling the electronic elements of the distal tip 118. For example, the actuator(s) 136 may control image capture, lighting levels, or other aspects of the imaging device 120 and/or the lighting elements 122. The handle 112 may also include one or more valves 138 for delivering air, water, etc. to the distal tip 118 and/or applying suction or negative pressure to the distal tip 118.

An umbilicus 139 may extend proximally from the handle 112. The umbilicus 139 may be used to couple the endoscope 110 to capital equipment, such as electronic controller(s), and/or source(s) of air, water, and/or suction. For example, the umbilicus 139 may be coupled to a fluid source connected to a fluid delivery portion configured to provide a fluid to a shaft 184, or at least a lumen of the shaft 184. Alternatively, the umbilicus 139 may be configured to receive both optical energy and/or fluid.

Although endoscopes are particularly referenced herein, the disclosure also encompasses other types of devices, such as duodenoscopes, bronchoscopes, gastroscopes, endoscopic ultrasound (“EUS”) scopes, colonoscopes, ureteroscopes, bronchoscopes, laparoscopes, cystoscopes, aspiration scopes, sheaths, catheters, or similar devices. A reference to endoscopes herein should be understood to encompass any of the above medical devices or any other similar medical device.

The first medical instrument 160 may include a handle 176 and a shaft 140. The shaft 140 may extend from a proximal end 142 to a distal end 144. The shaft 140 may be external to the endoscope 110, such that the shaft 140 may be external to, and extend alongside of, an outer surface of the shaft 116 of the insertion portion 114. The shaft 140 may have a tubular shape and may be flexible. For example, the shaft 140 may be formed from a polymer or polymer-like material (e.g., via extrusion).

The first medical instrument 160 may include a first grasping device 164 (e.g., a first end effector) and a second grasping device 166 (e.g., a second end effector). The first grasping device 164 and the second grasping device 166 may have any of the features of any medical device known in the art used to grasp tissue, for example, a forceps, grasper, or other type of medical device having jaws. The first grasping device 164 may include a pair of jaws 165 and the second grasping device 166 may include a pair of jaws 167.

The jaws 165 and the jaws 167 may consist of two opposing arms and these arms may be hinged at one end (e.g., a proximal end) to allow them to open and close, providing adjustable tension for gripping. The jaws 165 and the jaws 167 may be equipped with protrusions or gripping elements for grasping an object or tissue. In an embodiment, the jaws 167 may be smaller than the jaws 165. Alternatively or additionally, the second grasping device 166 may be smaller than the first grasping device 164 in other ways. The jaws 167 of the second grasping device 166 may be designed with for example, the gripping elements that avoid causing damage to the surrounding tissue. In another embodiment, the gripping elements may be distributed over an increased surface area of the gripping elements to distribute the gripping force over a larger area of tissue.

In an embodiment, one of the first grasping device 164 or the second grasping device 166 of the first medical instrument 160 may anchor the shaft 140 in a desired location, such that the other of the first grasping device 164 or the second grasping device 166 of the first medical instrument 160 may grasp a lesion. For example, the first grasping device 164 may be the primary grasping device to create traction on a target tissue by actuating the jaws 165 during a procedure, for example, endoscopic submucosal dissection (ESD).

The first grasping device 164 may be on the distalmost end of the distal end 144 of the shaft 140. The position of the second grasping device 166 may be proximal to that of the first grasping device 164. The second grasping device 166 may grab onto nearby tissue (e.g., tissue near to a target tissue) to provide stabilization of the first medical instrument 160. The second grasping device 166 may be positioned on a radially outer surface of the shaft 140 (e.g., on a distal portion of the shaft 140). In other words, the second grasping device 166 may extend radially outward from the shaft 140 (e.g., perpendicularly to a central longitudinal axis of shaft 140). In an example, the second grasping device 166 may be fixed with respect to the shaft 140 and couple directly to the shaft 140. Alternatively, second grasping device 166 may have a separate (e.g., second) shaft, and second grasping device 166 may be at a distalmost end of the separate shaft. The separate shaft may extend through at least a portion of shaft 140 and through an opening on a radially outer wall of shaft 140.

A handle 176 may be disposed at a proximal end of first medical instrument 160. The handle 176 may include a handle body 177 and one or more actuators 178. The actuator(s) 178 may be used for controlling aspects of the first medical instrument 160, for example, the first grasping device 164 and the second grasping device 166. For example, the actuator 178 may be a spool that is longitudinally movable along the handle body 177. The handle 176 may have any of the features of any medical device handle known in the art. The handle 176 may include additional and/or alternative actuators. A configuration of the handle 176 is merely exemplary and shown for illustrative purposes.

In an embodiment, the actuator 178 may be divided into two distinct actuators or spools, a first actuator 179A and a second actuator 179B. The first actuator 179A and the second actuator 179B may actuate separately (e.g., opening and closing) the first grasping device 164 or the second grasping device 166.

In an embodiment, sliding the first actuator 179A distally may actuate the first grasping device 164 (e.g., open the jaws 165), and sliding the first actuator 179A proximally may actuate the first grasping device 164 (e.g., close the jaws 165). Sliding the second actuator 179B distally may actuate the second grasping device 166 (e.g., open the jaws 167), and sliding the second actuator 179B proximally may actuate the first grasping device 164 (e.g., close the jaws 165). Although sliding of actuators 179A, 179B is referred to above, it will be appreciated that actuators 179A, 179B may be actuated by alternative movements, depending on a type of actuator used. In examples, a first wire may extend through the shaft and couple to the first grasping device 164 and be actuated by the first actuator 179A. A second wire may extend through the shaft and couple to the second grasping device 166 and be actuated by the second actuator 179B. The first actuator 179A and/or the second actuator 179B may incorporate an adjustable tension mechanism. For example, as first actuator 179A or second actuator 179B slides distally, the force applied to the tissue may increase. An operation of the handle 176 and the first medical instrument 160 are described below.

In some aspects, a second medical instrument 180 may extend through a working channel of the insertion portion 114. For example, the second medical instrument 180 may extend through the port 126 and extend out of the distal opening 125 of the working channel 124. The second medical instrument 180 may include a handle 182, a shaft 184 extending distally from the handle 182, and an end effector 186. In some examples, the end effector 186 of the second medical instrument 180 may be an electrosurgical knife, and may be electrified and/or may inject a fluid (e.g., via an internal lumen). However, the end effector 186 of the second medical instrument 180 may be any other suitable type of instrument, such as another type of knife, a snare, or a needle. The second medical instrument 180 may be utilized (e.g., via the handle 182) in order to cut, resect, or otherwise treat a target tissue, for example, to separate the target tissue from a body lumen wall.

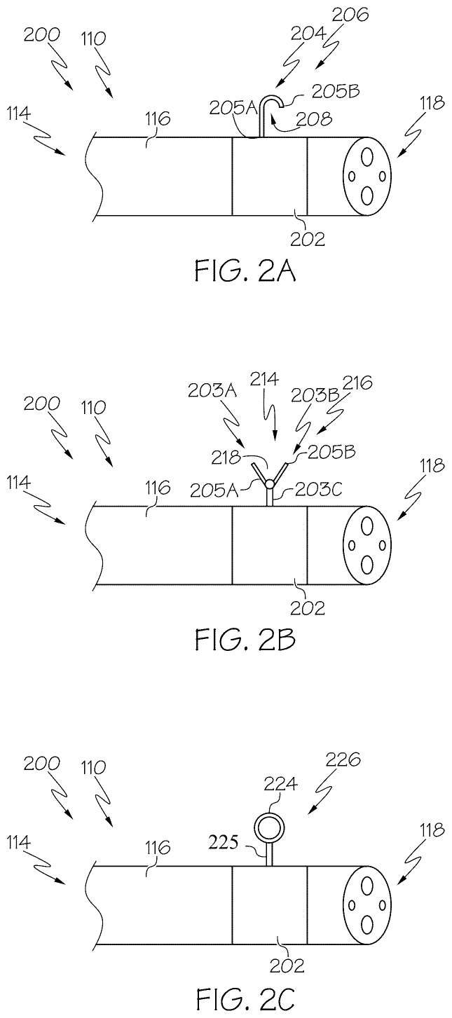

A method of using the system 100 in the configuration, as shown in FIG. 1, will now be described. The insertion portion 114 and the shaft 116 of the endoscope 110 may be inserted into the body lumen, such as the gastrointestinal tract, to access a target tissue area. The first medical instrument 160 may also be inserted into the body lumen. In some examples, the shaft 140 of the first medical instrument 160 may be coupled to the shaft 116 of the endoscope 110 (e.g., via the devices shown in FIGS. 2A-2C, discussed below).

The second grasping device 166 may be actuated using the actuator 179B, for example, to grab onto tissue proximate to the target tissue, thereby providing stability for the first medical instrument 160. Subsequently, the first grasping device 164 may be actuated using the first actuator 179A to grab onto a target tissue, applying traction to the target tissue. The second grasping device 166 may provide a purchase point for providing traction using the first grasping device 164. Once sufficient traction is applied to the target tissue, the second medical instrument 180 may be used to cut, resect, or otherwise treat the tissue as needed.

FIG. 2A depicts a system 200, including a distal portion of the shaft 116 of the insertion portion 114 and the endoscope 110 of FIG. 1. The medical system 200 may have any of the features of the medical system 100 described above, except where specified. The medical system 200 may include a guide device 206 that includes a coupler 202 and a hook 204 (a guide).

The coupler 202 may attach the guide device 206 to an outer (external) surface of the shaft 116. For example, the coupler 202 may extend around a portion of the shaft 116 near the distal tip 118. The coupler 202 may be configured to fit onto the shaft 116 of the insertion portion 114 of the endoscope 110 and the coupler 202 may extend radially around the shaft 116. Further, the coupler 202 may have dimensions compatible with an outer diameter of the shaft 116 to be configured to fit onto the shaft 116.

The following exemplary attachment mechanisms may be used to attach the coupler 202 to the shaft 116. In an embodiment, the coupler 202 may include internal threads compatible with external threads on the shaft 116. The threads may allow the coupler 202 to be movable along the length of the shaft 116, providing adjustable positioning. Once the coupler 202 is positioned, a locking mechanism may be included to secure the coupler 202 on the shaft 116. In another embodiment, the coupler 202 and the shaft 116 may include complementary snap-fit features that interlock when pressed together. The snap-fit connection may include locking tabs or features that engage with corresponding recesses on the shaft 116. In yet another embodiment, the coupler 202 may be pressed onto the shaft 116 until the interlocking components snap into place, securely holding the coupler 202 and the hook 204 in position. Further, the coupler 202 may be elastic or may have an inner opening that is sized so as to have an interference fit with the shaft 116.

Referring again to FIG. 2A, the hook 204 may be fixedly coupled to the coupler 202 (e.g., formed integrally with coupler 202 or otherwise be non-removably coupled to coupler 202) or be removably attached to the coupler 202 (e.g., using any of the attachment mechanisms described above). The hook 204 may be a curved or bent structure, including a rounded or curved end. For example, the hook 204 may include a fixed end 205A and a free end 205B. The fixed end 205A may be fixed to the coupler 202, and the free end 205B may be unattached to the coupler 202. The hook 204 may extend radially away from the fixed end 205A and then curve radially inward before terminating at the free end 205B. A gap 208 may extend between the free end 205B and the outer surface of the coupler 202.

The hook 204 may guide a shaft of an accessory instrument (e.g., having any of the properties of the medical instrument 160, including the shaft 140). The accessory instrument may be external to the shaft 116 of the endoscope 110. The accessory instrument may be any suitable type of accessory instrument having any suitable end effector (e.g., a clip, forceps, grasper, snare, knife, needle, ligation device, stent, stapler, etc.). The hook 204 may couple the shaft of the accessory instrument to the shaft 116 of the endoscope 110 or another medical device. Acting as a guide, the hook 204 may facilitate movement of the first medical instrument 160 along and/or with the shaft 116, allowing a user to guide an end effector (e.g., the first grasping device 164 and/or the second grasping device 166) to a desired position.

In some aspects, the hook 204 may facilitate navigating the endoscope 110 and the accessory instrument simultaneously to a tissue target site. Further, the hook 204 may allow a user to visualize an end effector of the accessory instrument while engaging tissue with the end effector (e.g., to apply traction to the tissue).

The gap 208 of the hook 204 allows the accessory instrument to be removed from (and/or introduced into) the guide device 206, decoupling the accessory instrument from the shaft 116 of the endoscope 110. Accordingly, after the accessory instrument is decoupled from the shaft 116, the user may maneuver the accessory instrument independently without being constrained by movement of the shaft 116 of the endoscope 110. For example, a user may use an end effector of the accessory instrument to grip and or grasp tissue (e.g., a target tissue or another tissue), and then a shaft of the accessory instrument may be guided through the gap 208 to decouple the accessory instrument from the shaft 116. In further examples, the accessory instrument may be reinserted through the gap 208 to perform other procedure aspects.

In some aspects, the accessory instrument may remain coupled to the shaft 116 via the guide device 206 throughout a procedure (e.g., during resection of a target tissue). This may allow for real-time adjustment and coordination between traction (e.g., applied by the accessory instrument) and lesion resection (e.g., applied by an instrument extended through a working channel 124 of the endoscope 110, such as the second medical instrument 180). For example, the user may adjust a traction applied by the accessory instrument based on the cutting progress of the second medical instrument 180 or other anatomical considerations.

FIG. 2B depicts a system 210 including distal portion of the shaft 116 of the insertion portion 114 and the endoscope 110 of FIG. 1 and a guide device 216. The system 210 may have any of the features or functions of the medical systems 100 and 200 described above, except where specified. In particular, the guide device 216 may have any of the properties and functions of the guide device 206, unless otherwise specified. In an embodiment, the guide device 216 may include the coupler 202 and a fork 214 (a guide).

The fork 214 may include two or more prongs 203A and 203B or tines extending outward from the coupler 202. In some examples, the fork 214 may also include a stem 203C that extends radially outward from the coupler 202 (e.g., approximately perpendicularly to a central longitudinal axis of the shaft 116). Each of the prongs 203A and 203B may have a fixed end 205A and a free end 205B. The fixed ends 205A may be fixed or coupled to stem 203C, while free ends 205B may not be coupled to another structure (e.g., stem 203C). A gap 218 may be defined between free ends 205B of prongs 204A and 203B. The gap 218 may extend along the length of the prongs 203A and 203B where they are not connected to any other structure. For example, from the tips of the free ends 205B to the point where the prongs connect to the stem 203C or coupler 202 (e.g., fixed ends 205A).

In some examples, the stem 203C may be omitted, and the fixed ends 205A and 205B may be coupled directly to the coupler 202. The prongs 203A and 203B may be angled apart from each other. In other words, the prongs 203A and 203B may extend away from one another between their respective fixed ends 205A and the free ends 205B. As shown in FIG. 2B, the prongs 203A and 203B may be substantially straight. Alternatively, the prongs 203A and 203B may be curved. In some examples, portions of the prongs 203A and 203B near the free ends 205B may be approximately parallel to one another.

The prongs 203A and 203B of fork 214 may guide an accessory device (e.g., having any of the properties of first medical instrument 160) by retaining the accessory device between the prongs 203A and 203B. The prongs 203A and 203B help the accessory device remain properly oriented and/or coupled to the shaft. Once the accessory device reaches the desired position within the body lumen, the accessory device may be maneuvered radially outward relative to the shaft 116 to be removed from the fork 214 through gap 218. Accordingly, the accessory device may disengage from the prongs 203A and 203B of the fork 214, and operate independently within the body lumen.

The guide device 216 may be used in any of the manners described above for guide device 206. For example, the guide device may be used during insertion of the shaft 116 into a body lumen to retain an accessory device alongside the shaft 116 and aid in insertion of the accessory device. The accessory device may be uncoupled from and/or coupled to the fork 214 during the course of a procedure, as desired.

FIG. 2C depicts a system 220 including distal portion of the shaft 116 of the insertion portion 114 and the endoscope 110 of FIG. 1 and a guide device 226. System 230 may have any of the features or functions of the medical systems 100, 200, and 210 described above, except where specified. In particular, the guide device 226 may have any of the properties and functions of the guide device 206 or the guide device 216, unless otherwise specified. In an embodiment, the guide device 216 may include the coupler 202 and a ring 224.

The ring 224 may be similar to hook 204, except for forming a complete loop or circle. The ring 224 may form a complete loop or circle around a shaft of an accessory instrument or another object to which it is attached. The ring 224 may be coupled to the coupler 202 by a stem 225, which may extend approximately perpendicularly to a longitudinal axis of shaft 116. Alternatively, stem 225aa may be omitted, and ring 224 may be directly coupled to the coupler 202.

Compared with the hook 204 and the fork 214, the ring 224 may lack a gap 208 or a gap 218, and thus an accessory instrument may not be removable from the ring 224. However, in some examples, the ring 224 may be frangible or may be actuated to create a gap (e.g., the gap 208) so that the accessory instrument may be removable from the ring 224.

In some examples, the ring 224 may have only a slightly larger inner diameter than the outer diameter of an accessory device, forming a snug fit around the accessory instrument shaft. In such examples, the accessory instrument may not be axially movable with respect to the ring 224 and the shaft 116. In such examples, movement imparted to the shaft 116 may impart the same movement to the shaft of the accessory instrument. Alternatively, the ring 224 may have an inner diameter such that the shaft of the accessory device is freely axially movable relative to the ring 224 and shaft 116. The relative inner diameter of the ring 224 and outer diameter of the shaft of the accessory instrument may be such that movement of the shaft 116 may generally impart the same movement to the shaft of the accessory instrument, but the accessory instrument may be separately movable with respect to the shaft 116.

For any of guide devices 206, 216, 226, the coupler 202 may be omitted, and the hook 204, fork 214, or ring 224 may be directly coupled to the shaft 116 (e.g., integrally formed with the shaft 116). Furthermore, multiple of guide devices 206, 216, 226 may be used in any combination on a given shaft 116 (e.g., to couple multiple accessory devices to the shaft 116).

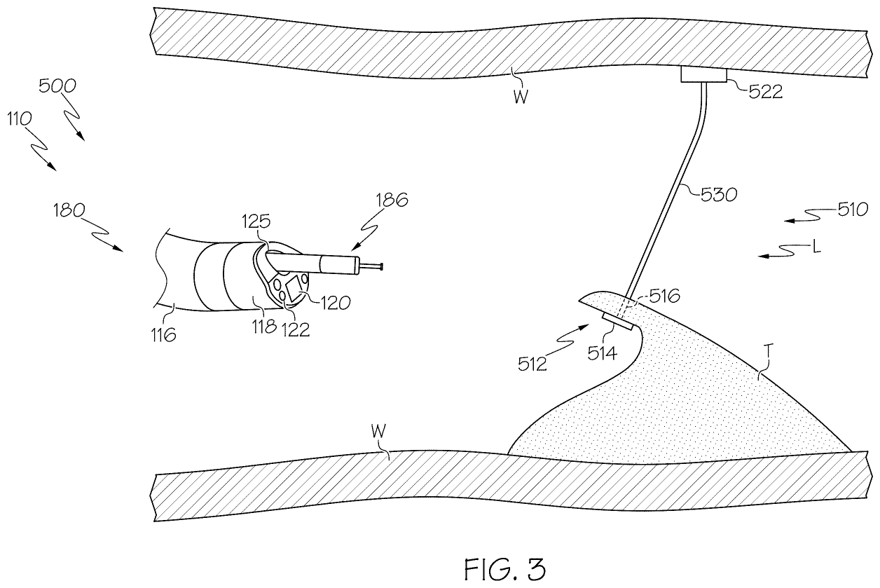

FIG. 3 depicts an alternative medical system 500. The medical system 500 may include the endoscope 110 and a medical device 510. The medical device 510 may include a first anchor 512, a second anchor 522, and a tether 530. For example, medical device 510 may apply traction to a target tissue “T.” In some examples, target tissue, “T” may include a lesion.

The first anchor 512 may include a horizontal bar 514 with a vertical stem 516, forming the shape of a T. With respect to this embodiment, the term “horizontal” refers to a direction that is approximately perpendicular to the tether 530, and the term “vertical” refers to a direction that is approximately parallel to the tether 530. The horizontal bar 514 may be implanted into a target tissue “T.” For example, when implanted, the horizontal bar 514 may be approximately parallel to an outer surface of target tissue “T.” The vertical stem 516 may be coupled to (e.g., directly attached to) the tether 530. As shown in FIG. 3, target tissue “T” may be in the process of being cut (e.g., by second medical instrument 180).

The second anchor 522 may be configured to be implanted into luminal wall “W” on an opposite side (or another side) of a bodily lumen “L” from the first anchor 512. The second anchor 522 may be a tag. The second anchor 522 may have any suitable shape. For example, the second anchor 522 may be approximately rectangular shaped. The second anchor 522 may have a loop or protrusion where the tether 530 may be affixed to the second anchor 522.

The tether 530 may be a string, wire, suture, thread, cable, band, or other member connecting the first anchor 512 and the second anchor 522. The first anchor 512 and the second anchor 522 may be coupled to one another via the tether 530. When the first anchor 512 and the second anchor 522 are coupled to target tissue “T” and luminal wall “W,” respectively, tension may be applied to target tissue “T.” In other words, a force may be applied on target tissue “T” toward the center of bodily lumen “L,” resulting in the lifting of target tissue “T.”

As shown in FIG. 3, the second medical instrument 180 (or another medical instrument) may be introduced into the bodily lumen “L” using the endoscope 110. Before, after, or concurrently while deploying the medical device 510 into the body lumen, the user may adjust the distal tip 118 of endoscope 110, such that the endoscope 110 (e.g., the end effector 186) may face target tissue “T.” The end effector 186 may cut or otherwise resect target tissue “T” away from a surrounding (e.g., adjacent) area of the luminal wall “W.” The traction applied to target tissue “T” by the medical device 510 may facilitate resection of target tissue “T” by lifting it away from surrounding tissue, as shown. As the target tissue “T” is resected, the medical device 510 may continue to pull the target tissue “T” toward the second anchor 522, improving visibility for a user and/or forming a tissue flap.

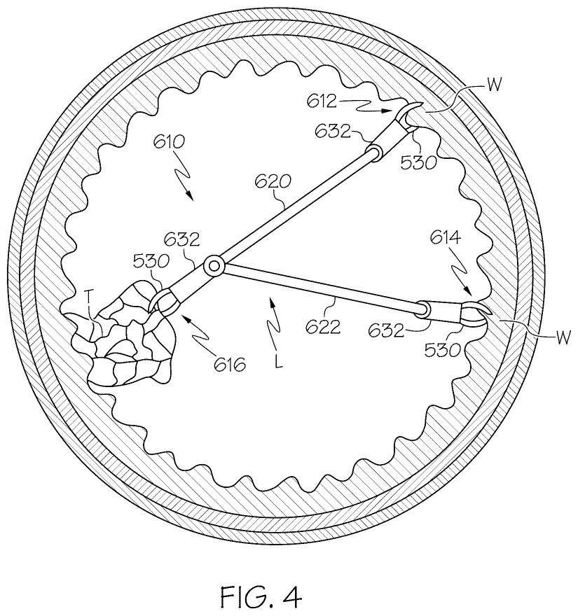

FIG. 4 depicts an alternative medical device 610 within a cross-section of a body lumen “L.” The medical device 610 may include a plurality of the anchors 612, 614, 616 and a plurality of the tethers 620, 622. The tethers 620, 622 may have any of the properties of the tether 530, unless otherwise specified. The anchors 612, 614, 616 may each be clips having any of the features of any medical clips known in the art. The anchors 612, 614, 616 may include jaws or arms having any of the properties of jaws 165 and 167, and configured to grip onto tissue of the bodily lumen “L.”

As shown in FIG. 4, the anchors 612, 614, and 616 may be separated from their respective delivery shafts (not shown) and deployed to tissue “T.” The anchors 612, 614, 616 may each include a capsule 632 configured to removably couple each of the anchors 612, 614, 616 to a shaft. In some examples, each of the anchors 612, 614, and 616 may be associated with a separate delivery shaft (not shown). In such examples, the shafts may be coupled together at proximal portions of the shafts. Each shaft may be associated with an individual handle (not shown), or the shafts (and the anchors 612, 614, 616) may be controlled with a single handle having multiple actuators.

The tether 620 may extend between the anchor 612 and the anchor 616, while the tether 622 may extend between the anchor 614 and the anchor 616. The tether 620 may include a wire, cable, band (e.g., elastic or inelastic band), or other structure. The tether 622 may have any of the properties of the tether 620. In some aspects, tethers 620 and 622 may have varying properties. For example, tether 620 may have a larger diameter and thickness compared to tether 622, while tether 622 may be thinner. Varying thicknesses of the tethers 620, 622 may cause a force to on the target tissue “T” to be directed more towards one or the other of the anchors 612, 614.

The tethers 620, 622 may be directly coupled to anchor 616. However, the configuration of tethers 620 and 622 is merely exemplary. In some configurations, a third tether may be coupled to anchor 616, and all of the tethers may be coupled together at a separate coupling point. In some examples, the third tether may have a smaller length than the tethers 620, 622.

A method of using the medical device 610 in the configuration, as shown in FIG. 4, will now be described. The anchor 616, which may be coupled to both of the tethers 620 and 622, may be affixed to the target tissue “T” within the bodily lumen “L.” The anchors 612 and 614 may be attached to portions of the luminal wall “W” that are situated across from the anchor 616, spanning at least a portion of the bodily lumen “L.”

The anchors 612 and 614 may exert a force on the target tissue “T,” pulling it towards the central axis of the bodily lumen “L” via the anchor 616. The force exerted by the anchors 612 and 614 may pull the target tissue toward the center of the lumen via the anchor 616, and away from the surrounding portions of the luminal wall “W.” As a result of the exerted force, the target tissue “T” is drawn towards the center of the bodily lumen “L.” Because both of the tethers 620 and 622 are directly coupled to the anchor 616, the force exerted on the target tissue “T” through the anchor 616 may be greater than the inward force exerted on tissues of luminal wall “W” grasped by the anchors 612 and 614. The pulling force on the target tissue “T” thereby facilitates resection of target tissue “T” pulling the target tissue “T” away from surrounding tissues of the luminal wall “W,” allowing for easier access during the resection process.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of these embodiments can be combined to achieve a same or similar result as one or more of the disclosed embodiments. Accordingly, the invention is not to be considered as limited by the foregoing description.

We claim:

1. A medical device comprising:
a shaft;
a first grasping device coupled to the shaft and including a first pair of jaws; and
a second grasping device coupled to the shaft and including a second pair of jaws.

2. The medical device of claim 1, wherein the first grasping device is on a distalmost end of the shaft.

3. The medical device of claim 1, wherein the second grasping device extends radially outward from the shaft.

4. The medical device of claim 1, wherein a handle is configured to actuate the first grasping device and the second grasping device.

5. The medical device of claim 4, wherein the handle includes a first actuator configured to actuate a first grasping device and a second actuator configured to actuate the second grasping device.

6. The medical device of claim 1, wherein a first wire extends through the shaft and couples to the first grasping device and a second wire extends through the shaft and couples to the second grasping device.

7. The medical device of claim 1, wherein the shaft is a first shaft, and wherein the second grasping device is further coupled to a distalmost end of a second shaft and wherein the second shaft extends through the first shaft.

8. The medical device of claim 1, wherein the first grasping device is configured to grasp a target tissue, and wherein the second grasping device is configured to grasp a wall of a body lumen.

9. The medical device of claim 7, wherein, in a configuration in which the first grasping device grasps a target tissue and the second grasping device grasps a wall of a body lumen, traction is applied to the target tissue.

10. A medical device including the medical device of claim 1, wherein the medical device is a first medical device, and the shaft is a first shaft, and wherein a system further includes a second medical device having a handle and a second shaft.

11. The medical device of claim 10, wherein the first shaft of the first medical device is configured to be external to the second shaft of the second medical device.

12. The medical device of claim 11, wherein the second medical device is a scope device.

13. The medical device of claim 10, wherein the second shaft includes a guide for coupling to the first shaft.

14. The medical device of claim 13, wherein the guide includes a ring, a prong, or a hook.

15. The medical device of claim 13, wherein the guide is coupled to a coupler, and wherein the coupler extends radially around the first shaft.

16. A medical device comprising:
a handle;
a first shaft; and
a guide extending radially outward from the first shaft, wherein the guide includes a ring, a hook, or a prong, and wherein the guide is configured to receive a second shaft of an accessory instrument.

17. The medical device of claim 16, wherein the guide further includes a coupler, wherein the coupler extends radially around an outer surface of the first shaft.

18. The medical device of claim 17, wherein the coupler is removably coupled to the first shaft.

19. A medical device comprising:
a first anchor, wherein the first anchor includes a stem and a bar, wherein the bar extends approximately perpendicularly to the stem, such that the anchor has a “T” shape;
a second anchor; and
a tether coupling the first anchor and the second anchor.

20. The medical device of claim 19, wherein the second anchor has a substantially rectangular shape.
